(19) United States
(12) Patent Application Publication
(10) Pub. No.: US 2022/0195542 A1
(43) Pub. Date: Jun. 23, 2022

(54) USE OF PRIMER PROBE COMBINATION AND KIT THEREOF IN HBV DETECTION

(71) Applicant: CHILDREN'S HOSPITAL OF CHONGQING MEDICAL UNIVERSITY, Chongqing (CN)

(72) Inventors: Yao ZHAO, Chongqing (CN); Yuting YANG, Chongqing (CN)

(73) Assignee: CHILDREN'S HOSPITAL OF CHONGQING MEDICAL UNIVERSITY, Chongqing (CN)

(21) Appl. No.: 17/599,578

(22) PCT Filed: Aug. 1, 2019
(86) PCT No.: PCT/CN2019/098794

§ 371 Date: Sep. 29, 2021

(30) Foreign Application Priority Data
Apr. 2, 2019 (CN) 201910259257.1

(51) Int. Cl. C12Q 1/70; C12Q 1/6818

(52) U.S. Cl. CPC C12Q 1/706; C12Q 1/6818

(57) ABSTRACT

Provided is the use of a primer and probe combination and a kit thereof in HBV detection. The primer and probe combination is selected from at least one of an S gene region primer and probe combination, a C gene region primer and probe combination, and an X gene region primer and probe combination. Primers and probes are respectively designed in conserved sequences of the S, C and X genomes of the hepatitis B virus, and a fluorescent quantitative PCR technique is used to simultaneously detect HBV DNA in the same tube.

### FIELD

The present disclosure relates to the field of biotechnology, specifically to use of a primer and probe combination and kit thereof for HBV detection.

### BACKGROUND

Hepatitis B virus (HBV) infection is one of the most harmful public health problems worldwide, and the total number of people infected with HBV in the world was as high as 2 billion. Since the discovery of hepatitis B virus infection, it has been receiving great attention. Especially in recent years, the potential clinical harm of chronic hepatitis B virus infection and the occult HBV infection (OBI) with low viral load has gradually been taken seriously, which greatly increases the risks of liver cirrhosis, primary liver cancer, liver failure, and HBV transmission, and approximately 1 million people die from diseases related to HBV infection each year. Although a highly effective hepatitis B vaccine has been available since 1982, there are still more than 400 million chronic carriers, of which 75% live in the Asia-Pacific region. Chronic hepatitis B virus infection in China is very serious, and HBV carriers and HBsAg-positive patients account for ⅓ of the patients in the world.

The gold standard for diagnosis of hepatitis B virus infection is the detection of HBV DNA, and quantitative detection of HBV DNA is very important for the treatment of patients with hepatitis B virus infection. At present, most of the hepatitis B virus nucleic acid detection kits mainly use one pair of primers and one probe to amplify and detect a relatively conservative gene fragment in the HBV genome. However, the replication of hepatitis B virus is based on a 3.5 kb pregenomic RNA as a template, and the negative-strand DNA of progeny virus is generated by reverse transcription by DNA polymerase. Due to the lack of automatic correction function of RNA reverse transcriptase, the replication of hepatitis B virus has the characteristics of high mutation rate. Long-term antiviral drug treatment (such as lamivudine, adefovir) in HBV patients may cause drug resistance mutation in the hepatitis B virus in the body. If the base mutation occurs in the target fragment of the amplification, it may cause the corresponding fragment to fail to be amplified normally, resulting in inaccurate quantification of HBV DNA, and even false negative results.

In general, the detection of hepatitis B surface antigen (HBsAg) in the blood is used clinically to determine whether there is HBV infection. However, with the development of molecular detection technology, it was first discovered in the 1980s that hepatitis B virus deoxyribonucleic acid (HBV DNA) may also be present in HBsAg-negative individuals, and this special form of chronic hepatitis B virus infection is called occult HBV infection (OBI).

With the deepening of research in recent years, the clinical harm of OBI has become more and more prominent. Many studies have found that occult hepatitis B virus infection is closely related to chronic liver disease, liver cirrhosis, hepatocellular carcinoma (HCC), etc. Occult hepatitis B virus can be transmitted through, for example, blood transfusion and organ transplantation; and if OBI patients receive immunosuppression treatment, the risk of hepatitis B virus reactivation will also increase. Therefore, timely and accurate detection of OBI patients is particularly important.

So far, HBV DNA detection has made great progress, but there is no standard, effective and uniform method for OBI detection. The European Association for the Study of the Liver (EASL) adopted the nested PCR technology as the diagnostic standard for OBI in 2007: extracting nucleic acid from the patient's liver tissue or serum/plasma samples, amplifying several gene sequences of HBV using multiple pairs of specific primers, and observing the amplified products by agarose gel electrophoresis. When the result shows that at least two different gene fragments are amplified positively (≥2), it indicates that OBI might exist. However, the nested PCR technology has the following disadvantages: (1) The operation is cumbersome, complicated, time-consuming and laborious; (2) It requires multiple pairs of primers to perform multiple PCR amplification reactions, which increases the chance of contamination and requires a large sample size; (3) The amplification result can only be qualitatively analyzed by agarose gel electrophoresis. Therefore, the nested PCR technology is not suitable for epidemiological investigation of OBI or large-scale clinical promotion and application.

The existing HBV detection methods have the following problems of: 1) inaccurate detection results caused by HBV mutations (for example, drug resistance mutations); 2) low sensitivity; 3) unable to meet the principle of at least two fragments showing positive results for OBI detection.

### SUMMARY

The purpose of the present disclosure is to provide a primer and probe combination, a kit comprising the primer and probe combination, and use thereof for HBV detection, which solves the problems in the prior art such as missed detection of HBV infection and false negative occurrence caused by low hepatitis B virus content in the body and proneness to base mutation, drug resistance mutation, and occult HBV infection.

In order to achieve the above and other related purposes, the first aspect of the present disclosure provides a primer and probe combination, which is selected from at least one of an S gene region primer and probe combination, a C gene region primer and probe combination, and an X gene region primer and probe combination.

The S gene region primer and probe combination is specifically selected from any one of the following combinations:

| | Combination S1: |
|---|---|
| | an upstream primer comprising a sequence |
| | as shown in |
| SEQ ID NO: 1 | |
| | (TTGCCCGTTTGTCCTCTAATTC), |
| | a downstream primer comprising a sequence |
| | as shown in |
| SEQ ID NO: 2 | |
| | (CATCCATAGGTTTTGTACAGCAAC), |
| | and |
| | a probe comprising a sequence as shown in |
| SEQ ID NO: 3 | |
| | (AGGATCATCAACCACCAGCACGGG); |
| | Combination S2: |
| | an upstream primer comprising a sequence |
| | as shown in |
| SEQ ID NO: 4 | |
| | (GTGTCTGCGGCGTTTATCA), |
| | a downstream primer comprising a sequence |
| | as shown in |
| SEQ ID NO: 5 | |
| | (CCCGTTTGTCCTCTAATTCCAG), |
| | and |
| | a probe comprising a sequence as shown in |
| SEQ ID NO: 6 | |
| | (TTCCTCTGCATCCTGCTGCTATGCC); |
| | and |
| | Combination S3: |
| | an upstream primer comprising a sequence |
| | as shown in |
| SEQ ID NO: 7 | |
| | (TGCCCGTTTGTCCTCTAATTCC), |
| | a downstream primer comprising a sequence |
| | as shown in |
| SEQ ID NO: 8 | |
| | (AGGTGCAGTTTCCATCCATAGG), |
| | and |
| | a probe comprising a sequence as shown in |
| SEQ ID NO: 9 | |
| | (TCATCAACCACCAGCACGGGACCA). |

The C gene region primer and probe combination is specifically selected from any one of the following combinations:

| | Combination C1: |
|---|---|
| | an upstream primer comprising a sequence |
| | as shown in |
| SEQ ID NO: 10 | |
| | (AGCCTTAAAATCTCCTGAGCATTG), |
| | a downstream primer comprising a sequence |
| | as shown in |
| SEQ ID NO: 11 | |
| | (CAAATTATTACCCACCCAGGTAGC), |
| | and |
| | a probe comprising a sequence as shown in |
| SEQ ID NO: 12 | |
| | (TCACCACACAGCACTCAGGCAAGC); |
| | Combination C2: |
| | an upstream primer comprising a sequence |
| | as shown in |
| SEQ ID NO: 13 | |
| | (AGGCAGGTCCCCTAGAAGAAG), |
| | a downstream primer comprising a sequence |
| | as shown in |
| SEQ ID NO: 14 | |
| | (ACATTGGGATTCCCGAGATTGAG), |
| | and |
| | a probe comprising a sequence as shown in |
| SEQ ID NO: 15 | |
| | (ACTCCCTCGCCTCGCAGACGAAGG); |
| | and |
| | Combination C3: |
| | an upstream primer comprising a sequence |
| | as shown in |
| SEQ ID NO: 16 | |
| | (ATCAACACTTCCGGAAACTACTG), |
| | a downstream primer comprising a sequence |
| | as shown in |
| SEQ ID NO: 17 | |
| | (TTCCCGAGATTGAGATCTTCTGC), |
| | and |
| | a probe comprising a sequence as shown in |
| SEQ ID NO: 18 | |
| | (GGCAGGTCCCCTAGAAGAAGAACT). |

The X gene region primer and probe combination is specifically selected from any one of the following combinations:

Combination X1:
an upstream primer comprising a sequence
as shown in
SEQ ID NO: 19
(TGCACTTCGCTTCACCTCTG),
a downstream primer comprising a sequence
as shown in
SEQ ID NO: 20
(TTGCTGAAAGTCCAAGAGTCCTC),
and
a probe comprising a sequence as shown in
SEQ ID NO: 21
(CGCATGGAGACCACCGTGAACGCC);
Combination X2:
an upstream primer comprising a sequence
as shown in
SEQ ID NO: 22
(ACTTCGCTTCACCTCTGCAC),
a downstream primer comprising a sequence
as shown in
SEQ ID NO: 23
(AGGTCGGTCGTTGACATTGC),
and
a probe comprising a sequence as shown in
SEQ ID NO: 24
(AGACCACCGTGAACGCCCACCG);
and
Combination X3:
an upstream primer comprising a sequence
as shown in
SEQ ID NO: 25
(CACCTCTCTTTACGCGGACTC),
a downstream primer comprising a sequence
as shown in
SEQ ID NO: 26
(AGTCCTCTTATGCAAGACCTTGG),
and
a probe comprising a sequence as shown in
SEQ ID NO: 27
(TGCCTTCTCATCTGCCGGACCGTG);

Each of the above probes may comprise a fluorescent dye and a fluorescence quencher.

Preferably, the S gene region primer and probe combination, the C gene region primer and probe combination, and the X gene region primer and probe combination are designed according to the highly conserved gene sequence of the hepatitis B virus standard strains registered in GenBank (accession numbers: X02763, D00329, X04615, X65259, X75657, X69798, AF160501, and AY090454).

The primers provided by the present disclosure have strong specificity, good sensitivity, and high detection efficiency, and can be used for effective amplification of HBV, thereby realizing high-efficiency detection and accurate quantification of HBV

Preferably, the fluorescent dye is selected from at least one of VIC, FAM, HEX, Cy5, Rox, and TET.

Preferably, the fluorescence quencher is selected from at least one of BHQ-1, BHQ-2, BHQ-3, BBQ, and TAMRA.

The detailed description of the above-mentioned fluorescent dyes and fluorescence quenchers is as follows:

### Fluorescent Dyes:

VIC [VIC: green fluorescent protein (GFP, a luminescent protein derived from a marine organism Aequoria Victoria)], the abbreviation of VIC may be derived from Victoria;

FAM: 6-Carboxy-fluorescein;

HEX: 5-Hexachloro-flurescein;

Cy5: Indodicarbocyanine;

Rox: Carboxy-x-rhodamine;

TET: 5-Tetrachloro-fluorescein;

### Fluorescence Quenchers:

BHQ-1, BHQ-2, BHQ-3: Black Hole Quencher-1, Black Hole Quencher-2, Black Hole Quencher-3;

BBQ: Black Berry Quencher;

TAMRA: Tetramethyl-6-carboxyrhodamine.

The above-mentioned fluorescent dyes and fluorescence quenchers are only a partial list, and other reagents with similar functions can also be used.

The above-mentioned primer and probe combinations can be combined and used for singlet single-gene, duplex double-gene, or triplex triple-gene hepatitis B virus detection.

The second aspect of the present disclosure provides use of the above-mentioned primer and probe combinations in the preparation of a hepatitis B virus detection reagent.

Preferably, the hepatitis B virus detection reagent includes singlet single-gene, duplex double-gene, triplex triple-gene quantitative in a single tube and/or qualitative detection reagent. The triplex triple-gene quantitative detection reagent in a single tube has the highest sensitivity and the best specificity.

The third aspect of the present disclosure provides a kit comprising the above-mentioned primer and probe combination.

Preferably, the kit further comprises a fluorescent quantitative reaction solution.

Preferably, the fluorescent quantitative reaction solution comprises buffer, dNTPs, and DNA polymerase. The fluorescent quantitative reaction solution can be purchased from the market, and specifically may be, for example, the 2×PCR Probes Master fluorescent quantitative reaction solution purchased from Roche (Switzerland). Of course, it is not limited to the above company, and the fluorescent quantitative reaction solution can also be purchased from companies such as ABI and BIO-RAD (USA).

Preferably, the volume of the fluorescence quantitative reaction solution is 5 μL-10 μL.

Preferably, the 681 nucleotides of the hepatitis B virus S gene region is shown in SEQ ID NO: 28.

Preferably, the 552 nucleotides of the hepatitis B virus C gene region is shown in SEQ ID NO: 29.

Preferably, the 465 nucleotides of the hepatitis B virus X gene region is shown in SEQ ID NO: 30.

According to the different purposes, the selected conserved regions may not be the same. The above three sequences are the conserved sequences obtained through comparison and screening, which are used for designing the primers and probes of the present disclosure.

Preferably, the kit further comprises at least one of a template, a positive control and a negative control.

Preferably, the template is selected from any one of a standard positive template and a human genomic DNA extract.

Preferably, the standard positive template is a recombinant plasmid.

Preferably, the standard positive template is a pLB-T vector plasmid carried a specific sequence of 3215 nucleotides of the whole hepatitis B virus genome.

Preferably, the concentration of the standard positive template is 5×10⁰˜5×10⁵ copies/μL, and the volume is 3.5 μL.

According to different purposes, the selected conserved regions may not be the same. The above two sequences are the conserved region sequences obtained through comparison and screening, which can be used for designing primers and probes.

Preferably, the positive control substance is hepatitis B virus DNA.

Preferably, the positive control substance is derived from hepatitis B virus isolated from the infected patient. Specifically, cloning and sequencing are performed, and the NCBI alignment tool is used for alignment. The positive control substance is nucleic acid of hepatitis B virus.

Preferably, the negative control substance is water.

Preferably, the concentrations of the upstream primer and the downstream primer are both 0.5 μM, and the volume is 0.125-0.35 μL.

Preferably, the concentration of the fluorescent probe is 0.2-0.5 μM, and the volume is 0.25-0.75 μL.

The fourth aspect of the present disclosure provides a method for detecting HBV by fluorescent quantitative PCR, comprising the following steps:

performing fluorescent quantitative PCR by using the fluorescent quantitative reaction solution, the standard positive template diluent and the above primer and probe combination, and then plotting a standard curve; and

performing fluorescent quantitative PCR by using the fluorescence quantitative reaction solution, the DNA from the sample to be tested and the primer and probe combination, and obtaining the HBV quantitative result of the sample according to the standard curve.

Preferably, the reaction program of the fluorescent quantitative PCR comprises:

1) Pre-denaturation: 95° C., 10 min; and

2) Amplification: denaturation, 95° C., 10 s; annealing, 62° C., 30 s; a total of 45 denaturation-annealing cycles.

The above programs are automatically completed by the Light Cycler 480 II fluorescent quantitative PCR machine of ROCHE (Switzerland), and the quantitative results are automatically calculated by the machine.

The present disclosure has at least one of the following beneficial effects:

1. The data show that the primer pairs provided by the present disclosure have strong specificity and high sensitivity, thereby realizing high-efficiency, sensitive and accurate quantification of HBV.

2. The probes for the S gene region, C gene region and X gene region respectively have different fluorescent labels and can be thus use in a single tube at the same time, which is convenient, rapid, and efficient, and reduces the costs of reagents and labor.

3. Simultaneous amplification of the conservative sequences of three HBV gene regions greatly reduces the occurrence of false negative results, improves the sensitivity of detection, and is suitable for detection of occult hepatitis B virus infection with low load.

4. The HBV DNA quantitative detection kit provided by the present disclosure has accurate quantification and can accurately and quantitatively detect viral nucleic acid; it has good sensitivity and specificity, and high efficiency; it has a short detection time of requiring only one hour, and it requires a total of only 2-3 hours including nucleic acid extraction; it has simple steps; and it enables the high-throughput sample detection at the same time.

5. The present disclosure can quantitatively detect HBV DNA, not only can detect different genes in separate tubes, but also can perform quantitative detection of multiple genes in a single tube at the same time.

6. The present disclosure is suitable for clinical or laboratory quantitative and qualitative detection of hepatitis B virus infection, early diagnosis of hepatitis B virus infection, monitoring and prediction of hepatitis B virus prevalence, and monitoring and evaluation of curative effects.

### BRIEF DESCRIPTION OF DRAWINGS

FIG. 1 is a schematic diagram showing the amplification curve for detecting the HBV S gene region in Example 1 of the present disclosure.

FIG. 2 is a schematic diagram showing the amplification curve for detecting the HBV C gene region in Example 2 of the present disclosure.

FIG. 3 is a schematic diagram showing the amplification curve for detecting the HBV X gene region in Example 3 of the present disclosure.

FIG. 4 is a diagram showing the program setting of duplex double-gene fluorescence quantitative PCR detection in a single tube in Example 4 of the present disclosure.

FIG. 5 is a schematic diagram showing the amplification curve for detecting the HBV S gene region in Example 4 of the present disclosure.

FIG. 6 is a schematic diagram showing the amplification curve for detecting the HBV C gene region in Example 4 of the present disclosure.

FIG. 7 is a schematic diagram showing the amplification curve for detecting the HBV S gene region in Example 5 of the present disclosure.

FIG. 8 is a schematic diagram showing the amplification curve for detecting the HBV X gene region in Example 5 of the present disclosure.

FIG. 9 is a diagram showing the program setting of simultaneous detection for triple-channel fluorescence in Example 6 of the present disclosure.

FIG. 10 is a schematic diagram showing the amplification curve for detecting the HBV S gene region in Example 6 of the present disclosure.

FIG. 11 is a schematic diagram showing the amplification curve for detecting the HBV C gene region in Example 6 of the present disclosure.

FIG. 12 is a schematic diagram showing the amplification curve for detecting the HBV X gene region in Example 6 of the present disclosure.

FIG. 13A is a diagram showing the amplification result of the S gene region in Example 8 of the present disclosure.

FIG. 13B is a diagram showing the amplification result of the C gene region in Example 8 of the present disclosure.

FIG. 13C is a diagram showing the amplification result of the X gene region in Example 8 of the present disclosure.

### DETAILED DESCRIPTION

The embodiments of the present disclosure will be further described below through specific examples, and those skilled in the art can easily understand other advantages and effects of the present disclosure from the content disclosed in this specification. The present disclosure can also be implemented or applied through other different specific embodiments, and various details in this specification can also be modified or changed based on different viewpoints and applications without departing from the spirit of the present disclosure.

Hepatitis B virus infection has been confirmed to be tightly related to the occurrence and development of diseases such as liver cirrhosis and primary liver cancer, and may also increase related clinical harm, such as the transmission of HBV through blood transfusion and organ transplantation.

At present, the detection of HBV DNA is the gold standard for the diagnosis of hepatitis B virus infection, and is mainly performed by real-time fluorescent quantitative PCR detection with specific primers for the conserved region of HBV gene. However, the existing detection kits generally only amplify one gene fragment, and if the target fragment has mutations, it may lead to false negative results.

In order to avoid false negative results, it is necessary to establish a method to detect different HBV genes simultaneously, so that the detection and determination of HBV infection will not be affected by the false negative results due to the mutation in a target detection fragment.

Therefore, in order to solve this problem, it is necessary to establish a highly sensitive HBV detection method, and multiple target genes can be set for detection at the same time, which can avoid gene mutation and detection escape, and can also detect the presence of HBV infection with high sensitivity.

The fluorescent quantitative PCR has the advantages of strong specificity, high sensitivity, good reproducibility, accurate quantification, fast speed, and fully enclosed reaction. It has been widely used in research fields such as molecular biology and medicine. Besides, compared with conventional PCR, it has the characteristics of stronger specificity, capable of effectively solving the contamination problem in PCR, and high degree of automation, so it has been widely used in scientific research and clinical diagnosis.

The present disclosure utilizes the advantages of fluorescent quantitative PCR, and provides a method of a multiplex multiple-gene (triplex triple-gene) fluorescent quantitative PCR detection in a single tube, which can solve the following problems in the existing HBV detection method:

1. Avoiding inaccurate results caused by HBV drug-resistant mutations in the HBV DNA. If the drug-resistant mutation occurs in one gene of HBV, there are still two more gene fragments that can be detected.

2. Overcoming the problem of low sensitivity of the existing HBV detection. If the copy number of the virus after HBV infection is not high, and there are differences in the copy number between genes, multiple detection will increase the detection possibility.

3. Overcoming the problem of being unable to meet the requirements of occult HBV infection (OBI) detection for positive fragments. For the principle of at least two positive fragments required for OBI detection, multiplex multiple-gene detection can fully follow it.

Based on the above, the present disclosure provides a triplex triple-gene HBV DNA detection method.

The following factors have also been considered:

1. Economics of detection: without increasing the detection cost too much by detection of multiple genes in a single tube.

2. Convenience of detection: without adding too many detection steps by detection of multiple genes in a single tube.

3. Accuracy of detection: without reducing the accuracy of detection by fluorescence quantitative PCR method.

4. Timeliness of detection: without increasing the time of detection by detection of multiple genes in a single tube.

5. Sample size for detection: without increasing the amount of serum required for detection by detection of multiple genes in a single tube.

6. Complexity of detection: without requiring additional training of detection operators by same fluorescence quantitative PCR method.

7. Sensitivity of detection: a higher detection sensitivity by simultaneous detection of multiple genes.

The present disclosure adopts multiple pairs of primer and probe combination, not only can detect different genes in a single tube, but also can detect the conservative sequences of different HBV genes in a single tube at the same time by using the fluorescent quantitative PCR method. The present disclosure can further improve the sensitivity of the HBV DNA detection method, obtain more accurate and effective quantitative results, and provide a more powerful basis for the formulation of clinical prevention or treatment regimens.

Main Experimental Materials:

DNA extraction kit (QIAamp Viral DNA Mini Kit) purchased from Qiagen; 2×PCR Probes Master (fluorescence quantitative reaction solution, specification 5 ml) purchased from Roche, cat. No. 04707494001; Escherichia coli DH5a, plasmid extraction kit, purchased from Tiangen BioTech (Beijing) Co., Ltd.

Primers and probes. According to the standard sequences of hepatitis B virus in GenBank (accession number: X02763, D00329, X04615, X65259, X75657, X69798, AF160501, AY090454), CLUSTAL X multiple sequence alignment software based on Smith Waterman algorithm was used for alignment and analysis, and MEGA5.0 software, Oligo6.71 software were used for analysis to find out conserved specific regions that can be used for designing primers and probes. A pair of specific primer and a specific TaqMan probe were designed according to the conservative sequences of the S gene region, C gene region and X gene region of HBV DNA respectively to establish a method of multiplex multiple-gene fluorescent quantitative PCR detection for hepatitis B virus in a single tube with high sensitivity and specificity, which can quickly and accurately detect HBV DNA load. The primers and probes were synthesized by Shanghai Invitrogen Life Technologies Co., Ltd.

Standard positive template is pLB-T vector plasmid inserted with a specific sequence of the whole genome of hepatitis B virus (the plasmid extraction kit used was the pLB zero background rapid cloning kit, purchased from Tiangen BioTech Co., Ltd.). Specifically, the whole genomic fragment of hepatitis B virus was cloned into the vector, which was then transformed into Escherichia coli DH5a for amplification, and then plasmid DNA was extracted with a plasmid extraction kit. The plasmid DNA was then quantified by a spectrophotometer A₂₆₀ and diluted to 1×10⁸ copies/μL.

The negative control was water.

The positive control substance was HBV DNA, which was derived from the HBV isolated from patient, cloned and sequenced, and aligned with NCBI gene tools to obtain the positive control hepatitis B virus nucleic acid.

The sequences used in the following examples are as follows:

| | | | |
|---|---|---|---|
| Combinations | S1 | Primer | TTGCCCGTTTGTCCTCTAATTC |
| for S Gene | | (5′-3′) | CATCCATAGGTTTTGTACAGCA |
| | | | AC |
| | | Probe | VIC-TCATCAACCACCAGCACG |
| | | | GGACCA-BHQ1 |
| | S2 | Primer | GTGTCTGCGGCGTTTATCACCC |
| | | (5′-3′) | GTTTGTCCTCTAATTCCAG |
| | | Probe | VIC-TTCCTCTGCATCCTGCTG |
| | | | CTATGCC-BHQ1 |
| | S3 | Primer | TGCCCGTTTGTCCTCTAATTCC |
| | | (5′-3′) | AGGTGCAGTTTCCATCCATAGG |
| | | Probe | VIC-AGGATCATCAACCACCAG |
| | | | CACGGG-BHQ1 |
| Combinations | C1 | Primer | AGCAACACTTCCGGAAACTACT |
| for C Gene | | (5′-3′) | GTTCCGGAGATTGAGATCTTCA |
| | | | GG |
| | | Probe | FAM-GGTAGTCCCTTAGAAGAA |
| | | | GAAC-BHQ1 |
| | C2 | Primer | AGGCAGGTCCCCTAGAAGAAGA |
| | | (5′-3′) | CATTGGGATTCCCGAGATTGAG |
| | | Probe | FAM-ACTCCCTCGCCTCGCAGA |
| | | | CGAAGG-BHQ1 |
| | C3 | Primer | AGCCTTAAAATCTCCTGAGCAT |
| | | (5′-3′) | TGCAAATTATTACCCACCCAGG |
| | | | TAGC |
| | | Probe | FAM-TCACCACACAGCACTCAG |
| | | | GCAAGC-BHQ1 |
| Combinations | X1 | Primer | TGCACTTCGCTTCACCTCTGTT |
| for X Gene | | (5′-3′) | GCTGAAAGTCCAAGAGTCCTC |
| | | Probe | CY5-CGCATGGAGACCACCGTG |
| | | | AACGCC-BHQ3 |
| | X2 | Primer | ACTTCGCTTCACCTCTGCACAG |
| | | (5′-3′) | GTCGGTCGTTGACATTGC |
| | | Probe | CY5-AGACCACCGTGAACGCCC |
| | | | ACCG-BHQ3 |
| | X3 | Primer | CACCTCTCTTTACGCGGACTCA |
| | | (5′-3′) | GTCCTCTTATGCAAGACCTTGG |
| | | Probe | CY5-TGCCTTCTCATCTGCCGG |
| | | | ACCGTG-BHQ3 |

### Example 1

### Single S Gene Region Fluorescence Quantitative PCR for Quantitative Detection of HBV

1. The standard positive template was dissolved with distilled water, and diluted in series to 5×10⁵ copies/μL, 5×10⁴ copies/μL, 5×10³ copies/μL, 5×10² copies/μL, 5×10¹ copy/μL, and 5×10⁰ copies/μL.

2. Fluorescence quantitative PCR (10 μL, of amplification reaction system): the fluorescence quantitative reaction solution 5.0 μL, forward primer of S gene region primer pair 0.35 μL (0.5 μM), reverse primer of S gene region primer pair 0.35 μL (0.5 μM), fluorescent probe 0.75 μL (0.2 μM). The primer and probe combination was selected from combination S1, and the standard positive template was 3.5 μL.

Fluorescence quantitative PCR reaction parameters were as follows:

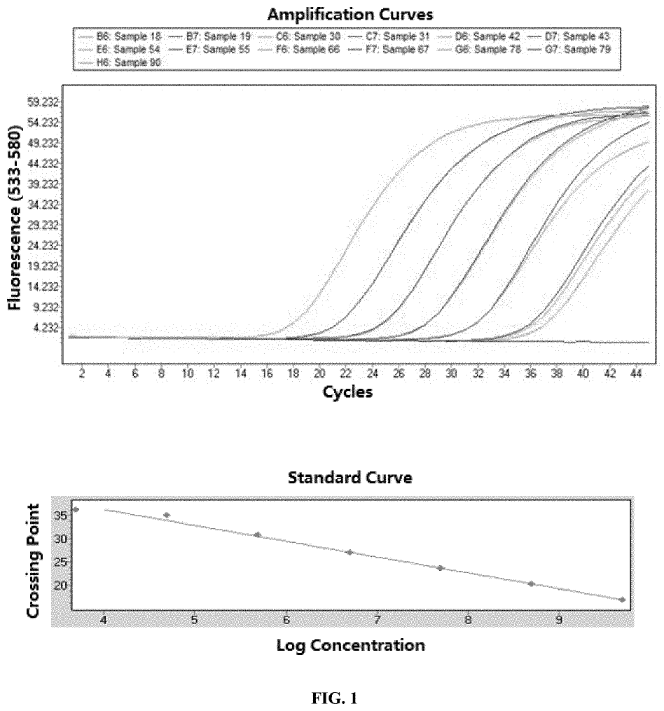

Pre-denaturation: 95° C., 10 min; denaturation: 95° C., 10 s; annealing: 62° C., 30 s; a total of 45 denaturation-annealing cycles. These steps were automatically completed by the Light Cycler 480 II fluorescent quantitative PCR machine (ROCHE, Switzerland), and the quantitative results were automatically calculated by the machine. The results are shown in FIG. 1.

### Example 2

### Single C Gene Region Fluorescence Quantitative PCR for Quantitative Detection of HBV

Fluorescence quantitative PCR (10 μL of amplification reaction system): the fluorescence quantitative reaction solution 5.0 μL, forward primer of C gene region primer pairs 0.35 μL (0.5 μM), reverse primer of C gene region primer pair 0.35 μL (0.5 μM), fluorescent probe 0.75 μL (0.2 μM). The primer and probe combination was selected from combination C1, and the standard positive template was 3.5 μL.

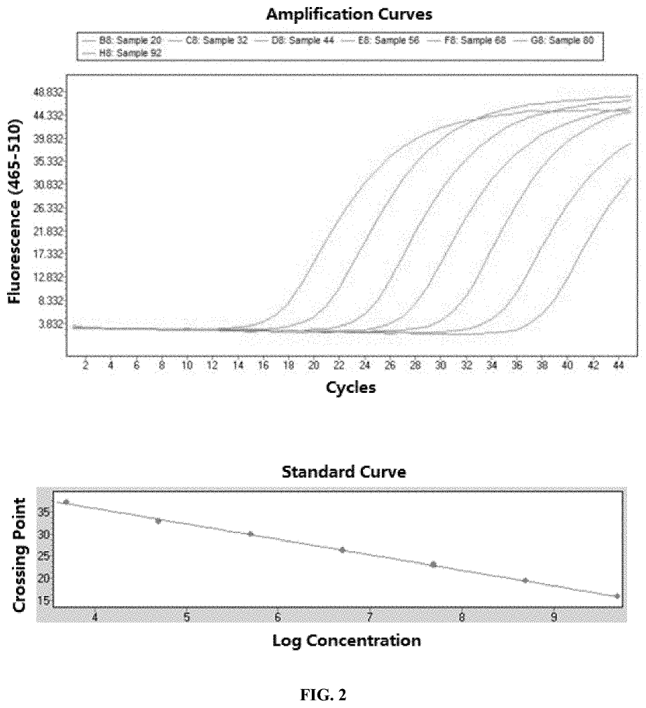

The fluorescence quantitative PCR reaction parameters were the same as in Example 1, and the results are shown in FIG. 2.

### Example 3

### Single X Gene Region Fluorescence Quantitative PCR for Quantitative Detection of HBV

Fluorescence quantitative PCR (10 of amplification reaction system): the fluorescence quantitative reaction solution 5.0 μL, forward primer of X gene region primer pairs 0.35 μL (0.5 μM), reverse primer of X gene region primer pairs 0.35 μL (0.5 μM), fluorescent probe 0.75 μL (0.2 μM). The primer and probe combination was selected from combination X1, and the standard positive template was 3.5 μL.

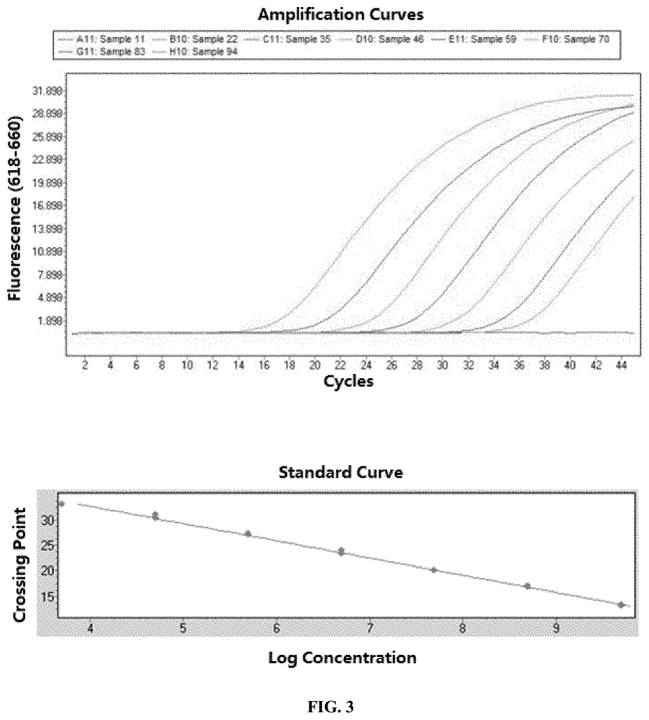

The fluorescence quantitative PCR reaction parameters were the same as in Example 1, and the results are shown in FIG. 3.

### Example 4

### Duplex Double-Gene Fluorescence Quantitative PCR for Quantitative Detection of HBV in the Same Tube

Fluorescence quantitative PCR (10 μL of amplification reaction system): the fluorescence quantitative reaction solution 5.0 μL, forward primer of S gene region primer pairs 0.175 μL (0.5 μM), reverse primer of S gene region primer pair 0.175 μL (0.5 μM), fluorescent probe 0.375 μL (0.2 μM), forward primer of C gene region primer pair 0.175 μL (0.5 μM), reverse primer of C gene region primer pair 0.175 μL (0.5 μM), fluorescent probe 0.375 μL (0.2 μM). The primer and probe combination was selected from combination S1 and C1, and the standard positive template was 3.5 μL.

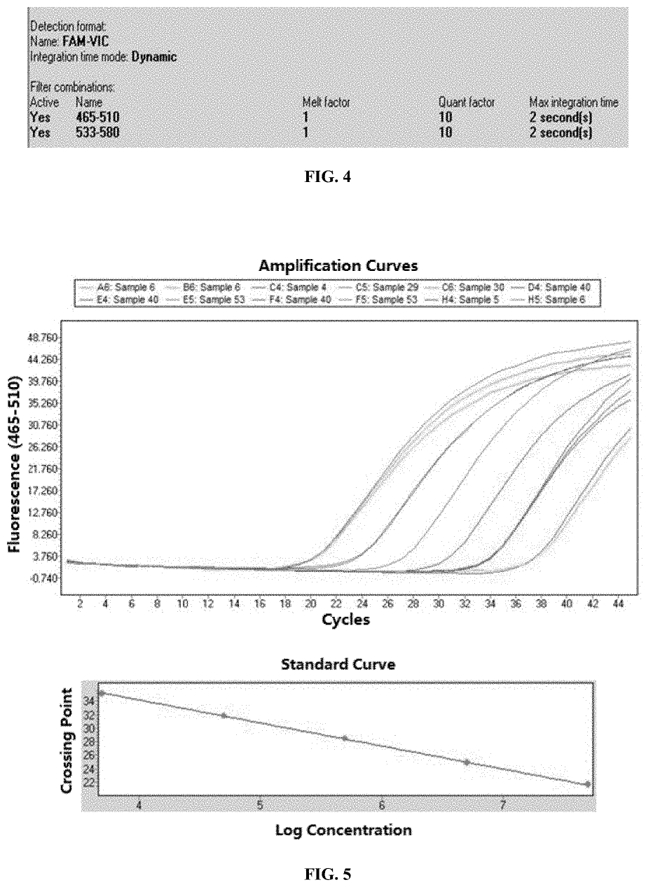

The fluorescence quantitative PCR reaction parameters were the same as in Example 1. Dual-channel fluorescence detection was performed, and the settings are shown in FIG. 4.

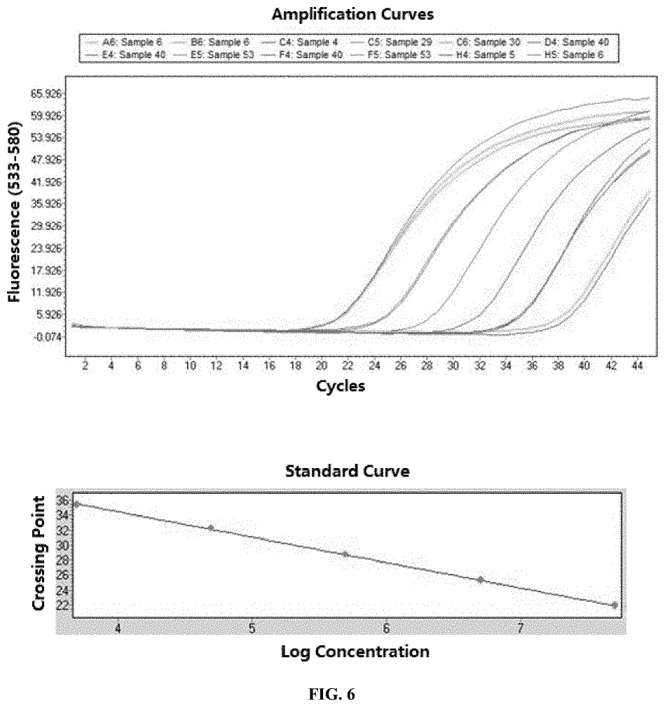

The amplification result of the S gene region is shown in FIG. 5, and the amplification result of the C gene region is shown in FIG. 6.

The experiment results show that as for both the amplification efficiency of the method of duplex double-gene PCR detection in a single tube and the amplification efficiency of singlet single-gene fluorescence quantitative PCR, the target fragment of each gene were amplified well without interference.

### Example 5

### Duplex Double-Gene Fluorescence Quantitative PCR for Quantitative Detection of HBV in the Same Tube

Fluorescence quantitative PCR (10 μL of amplification reaction system): the fluorescence quantitative reaction solution was 5.0 μL, forward primer of S gene region primer pair 0.175 μL (0.5 μM), reverse primer of S gene region primer pair 0.175 μL (0.5 μM), fluorescent probe 0.375 μL (0.2 μM), forward primer of X gene region primer pair 0.175 μL (0.5 μM), reverse primer of X gene region primer pair 0.175 μL (0.5 μM), fluorescent probe 0.375 μL (0.2 μM). The primer and probe combination was selected from combination S1 and X1, and the standard positive template was 3.5 μL.

The fluorescence quantitative PCR reaction parameters were the same as in Example 1 and Dual-channel fluorescence detection was performed.

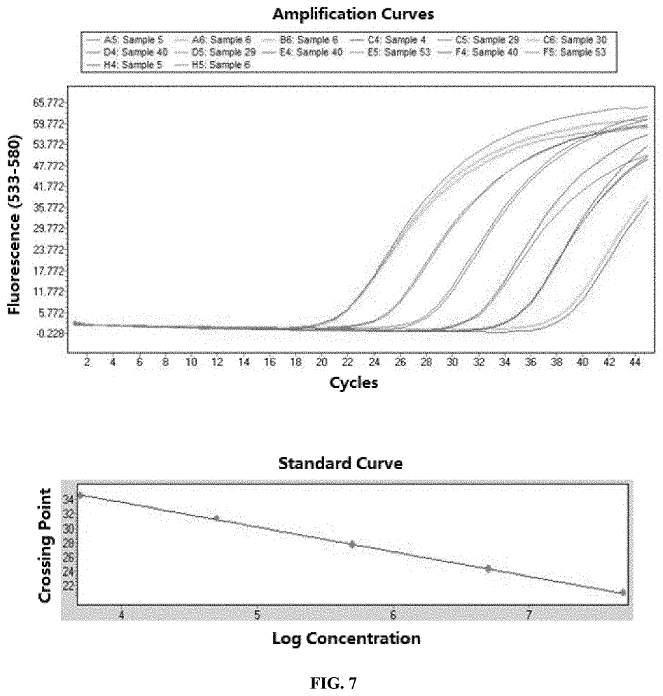

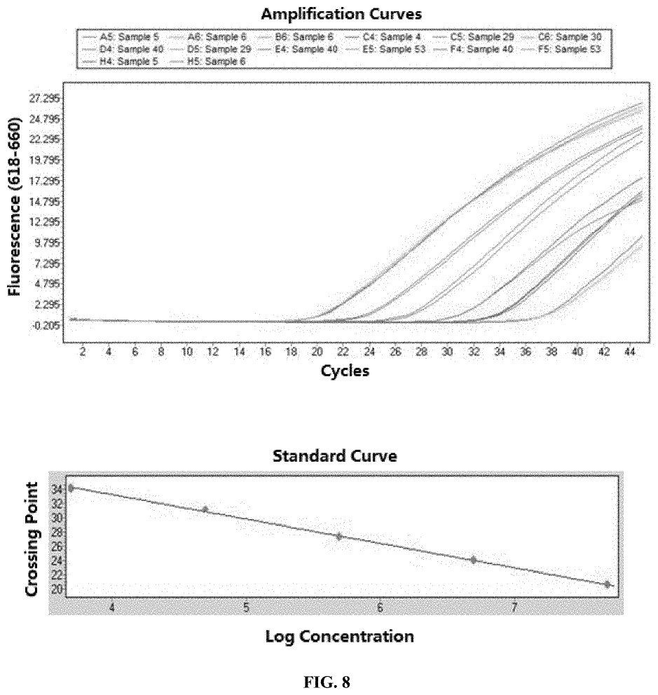

The amplification result of the S gene region is shown in FIG. 7, and the amplification result of the X gene region is shown in FIG. 8.

The experiment results show that the amplification efficiencies of the method of duplex double-gene PCR detection in a single tube, singlet single-gene fluorescence quantitative PCR, and triplex triple-gene fluorescent quantitative PCR in a single tube were basically the same, and the target fragment of each gene were amplified well without interference.

### Example 6

### Multiplex Multiple-Gene Fluorescence Quantitative PCR for Quantitative Detection of HBV in the Same Tube

Fluorescence quantitative PCR (10 of amplification reaction system): the fluorescence quantitative reaction solution 5.0 μL, forward primer of S gene region primer pair 0.125 μL (0.5 μM), reverse primer of S gene region primer pair 0.125 μL (0.5 μM), fluorescent probe 0.25 μL (0.2 μM), forward primer of C gene region primer pair 0.125 μL (0.5 μM), reverse primer of C gene region primer pair 0.125 μL (0.5 μM), fluorescent probe 0.25 μL (0.2 μM), forward primer of X gene region primer pair 0.125 μL (0.5 μM), reverse primer of X gene region primer pair 0.125 μL (0.5 μM), fluorescent probe 0.25 μL (0.2 μM). The S, C, X gene region primer and probe combination was selected from combination S1, C1, X1, respectively, and the standard positive template was 3.5 μL.

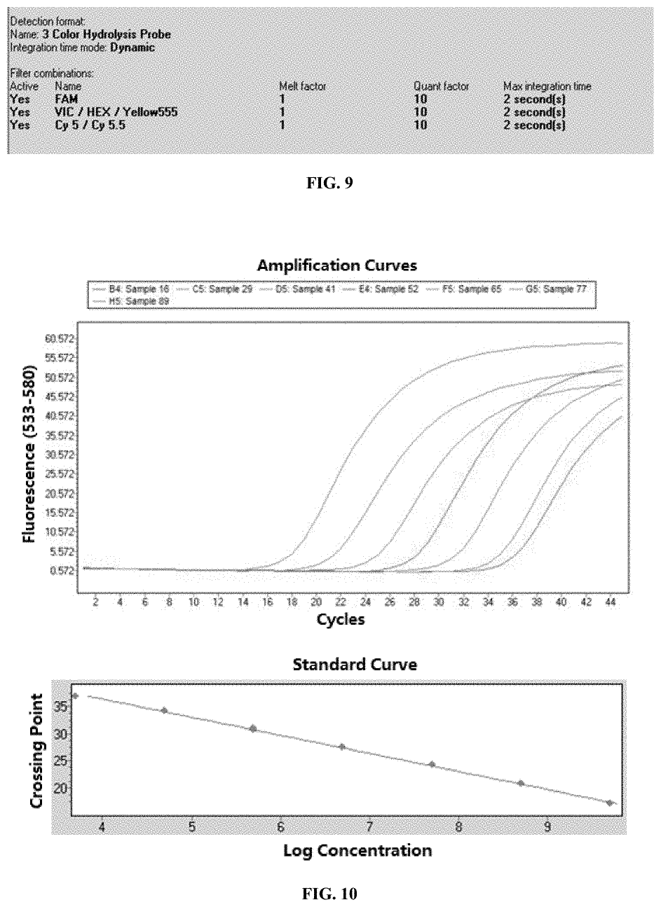

The fluorescence quantitative PCR reaction parameters were the same as in Example 1. Triple-channel fluorescence detection was performed and the settings are shown in FIG. 9.

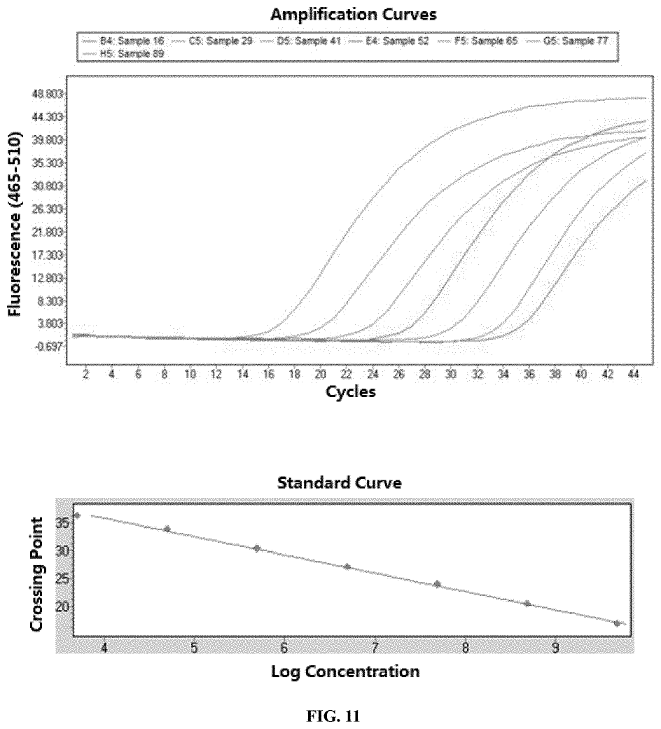

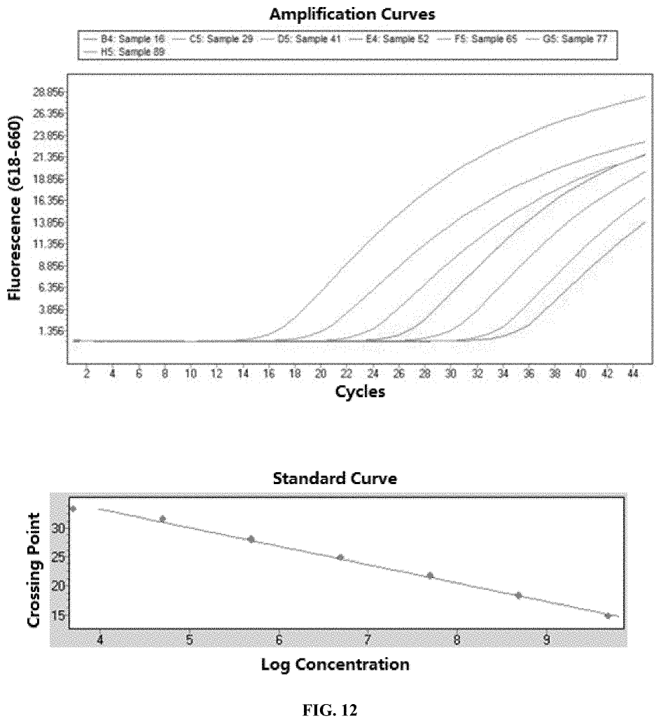

The amplification result of the S gene region is shown in FIG. 10, the amplification result of the C gene region is shown in FIG. 11, and the amplification result of the X gene region is shown in FIG. 12.

The experiment results show that the amplification efficiencies of the method of multiplex multiple-gene fluorescence quantitative PCR detection for occult hepatitis B virus in a single tube and singlet single-gene fluorescence quantitative PCR were basically the same, and the target fragment of each gene were amplified well without interference.

### Example 7

### Multiplex Multiple-Gene Fluorescence Quantitative PCR for Quantitative Detection of HBV in a Single Tube

Fluorescence quantitative PCR experiment (20 μL of amplification reaction system): the fluorescence quantitative reaction solution was 10.0 μL, forward primer of S gene region primer pair 0.25 μL (0.5 μM), reverse primer of S gene region primer pair 0.25 μL (0.5 μM), fluorescent probe 0.50 μL (0.2 μM), forward primer of C gene region primer pair 0.25 μL (0.5 μM), reverse primer of C gene region primer pair 0.25 μL (0.5 μM), fluorescent probe 0.50 μL (0.2 μM), forward primer of X gene region primer pair 0.25 μL (0.5 μM), reverse primer of X gene region primer pair 0.25 μL (0.5 μM), fluorescent probe 0.50 μL (0.2 μM). The S, C, X gene region primer and probe combination was selected from combination S1, C1 and X1, respectively, and the standard positive template was 7.0 μL.

The fluorescence quantitative PCR reaction parameters were the same as in Example 1. Triple-channel fluorescence detection was performed at the same time.

### Example 8

### Analysis of Specificity of Multiplex Multiple-Gene Fluorescence Quantitative PCR for Detection of HBV DNA in a Single Tube

In this example, the specificity of S, C, and X gene region primer and probe combinations was tested. The above multiplex fluorescence quantitative PCR was performed using human genomic DNA as a template in order to verify the specificity of the method.

Fluorescence quantitative PCR (10 of amplification reaction system): the fluorescence quantitative reaction solution 5.0 μL, forward primer of S gene region primer pair 0.125 μL (0.5 μM), reverse primer of S gene region primer pair 0.125 μL (0.5 μM), fluorescent probe 0.25 μL (0.2 μM), forward primer of C gene region primer pair 0.125 μL (0.5 μM), reverse primer of C gene region primer pair 0.125 μL (0.5 μM), fluorescent probe 0.25 μL (0.2 μM), forward primer of X gene region primer pair 0.125 μL (0.5 μM), reverse primer of X gene region primer pair 0.125 μL (0.5 μM), fluorescent probe 0.25 μL (0.2 μM). The S, C, X gene region primer and probe combination was selected from combination S1, C1, X1, respectively, and the human genomic DNA extract was used as a template, 3.5 μL.

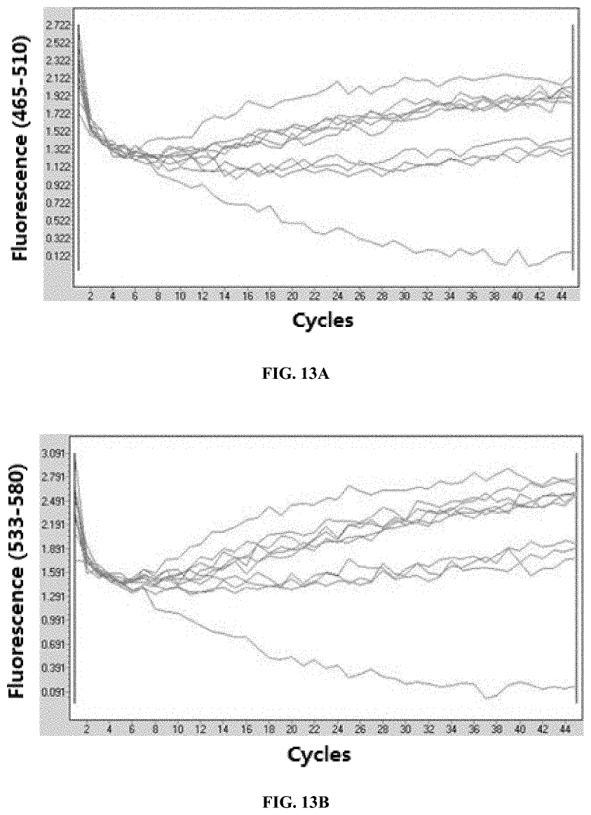

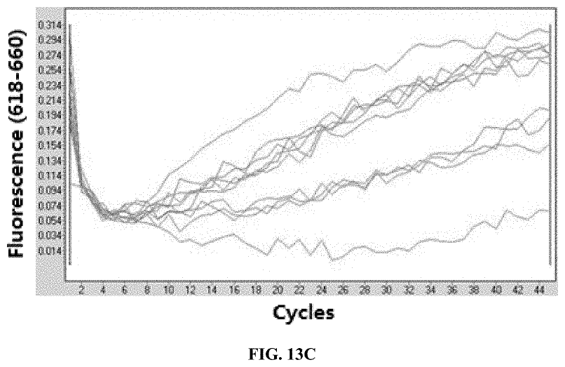

The fluorescence quantitative PCR reaction parameters were the same as in Example 1. Triple-channel fluorescence detection was performed and the results are shown in FIG. 13A, FIG. 13B and FIG. 13C. FIG. 13A shows the amplification curve of the S gene region, FIG. 13B shows the amplification curve of the C gene region, and FIG. 13C shows the amplification curve of the X gene region.

### Example 9

### Detection of Clinical Samples by Multiplex Multi-Gene Fluorescence Quantitative PCR

DNA extraction kit was used. According to the operating instructions of the kit, the DNA of the sample to be tested (serum/plasma samples were isolated from clinical samples from patients in the Department of Infectious Diseases) was extracted, and stored at 4° C. for later use.

Fluorescence quantitative PCR (10 of amplification reaction system): the fluorescence quantitative reaction solution 5.0 μL, forward primer of S gene region primer pair 0.125 μL (0.5 μM), reverse primer of S gene region primer pair 0.125 μL (0.5 μM), fluorescent probe 0.25 μL (0.2 μM), forward primer of C gene region primer pair 0.125 μL (0.5 μM), reverse primer of C gene region primer pair 0.125 μL (0.5 μM), fluorescent probe 0.25 μL (0.2 μM), forward primer of X gene region primer pair 0.125 μL (0.5 μM), reverse primer of X gene region primer pair 0.125 μL (0.5 μM), fluorescent probe 0.25 μL (0.2 μM). The S, C, X gene region primer and probe combination was selected from combination S1, C1, X1, respectively, and the template was 3.5 μL. The serial dilutions of standard positive template were used to plot standard curve, with the negative control substance water as negative control for PCR reaction, and the positive control substance as positive control for PCR reaction, in order to detect whether the PCR reaction system functioned well. Three replicates were set for the sample to be tested to ensure the accuracy and stability of the experiment. The fluorescence quantitative PCR reaction parameters were the same as in Example 1. Triple-channel fluorescence detection was performed.

By comparing the sample to be tested with the standard curve of the serial dilutions of standard positive template, the initial copy number of the sample to be tested was quantified. The detection results of samples 1-23 are shown in Table 1.

TABLE 1
Quantitative detection results of test samples

| | Concentration (copies/mL) | | |
|---|---|---|---|
| Sample No. | S Gene Region | C Gene Region | X Gene Region |
| Test sample 1 | 7.97 × 10⁴ | 1.04 × 10⁵ | — |
| Test Sample 2 | 4.74 × 10⁴ | 1.46 × 10³ | — |
| Test Sample 3 | 3.37 × 10⁴ | 6.67 × 10³ | — |
| Test Sample 4 | 1.51 × 10⁵ | 2.13 × 10⁵ | — |
| Test Sample 5 | 1.09 × 10⁶ | 1.30 × 10⁶ | 1.52 × 10⁶ |
| Test Sample 6 | 2.08 × 10⁵ | 2.33 × 10⁵ | 3.77 × 10³ |
| Test Sample 7 | 1.71 × 10⁵ | 2.49 × 10⁴ | 2.67 × 10³ |
| Test Sample 8 | 3.62 × 10⁵ | 2.04 × 10⁵ | — |
| Test Sample 9 | 4.65 × 10⁶ | 6.77 × 10⁶ | — |
| Test Sample 10 | 8.22 × 10⁵ | 1.26 × 10⁶ | — |
| Test Sample 11 | 4.28 × 10⁵ | 2.28 × 10⁵ | — |
| Test Sample 12 | 6.11 × 10⁶ | 8.40 × 10⁶ | 2.80 × 10⁶ |
| Test Sample 13 | 2.02 × 10⁵ | 2.33 × 10⁵ | 3.77 × 10³ |
| Test Sample 14 | 9.28 × 10⁵ | 3.49 × 10⁵ | 2.05 × 10² |
| Test Sample 15 | 1.07 × 10⁶ | 1.77 × 10⁶ | — |
| Test Sample 16 | 2.46 × 10⁵ | 6.88 × 10⁴ | 1.94 × 10⁴ |
| Test Sample 17 | 1.42 × 10⁷ | 5.30 × 10⁶ | 3.06 × 10⁴ |
| Test Sample 18 | 3.45 × 10⁸ | 6.23 × 10⁸ | 3.19 × 10⁸ |
| Test Sample 19 | 7.99 × 10⁸ | 1.79 × 10⁹ | — |
| Test Sample 20 | 9.35 × 10⁶ | 8.82 × 10⁵ | 8.19 × 10² |
| Test Sample 21 | 8.66 × 10⁸ | 1.21 × 10⁹ | 6.09 × 10⁸ |
| Test Sample 22 | 6.45 × 10⁸ | 5.94 × 10⁸ | — |
| Test Sample 23 | 1.38 × 10⁹ | 1.64 × 10⁹ | — |
| Negative Control | 0 | | |
| Positive Control | ≥500 | ≥500 | ≥500 |

From the experimental results in Table 1, it can be seen that after the PCR reaction, there is no detectable fluorescent signal in the negative control substance tube, while the hepatitis B virus with a copy number within the control range was detected in the positive control substance tube, indicating that the PCR reaction system functioned well.

By comparing with the standard curve, the hepatitis B virus copy number of the sample to be tested was determined, indicating the sample as hepatitis B virus positive. The specific value (such as 7.97×10⁴) represents the specific copy number of hepatitis B virus infected.

### Comparative Example 1

Detection Using Hepatitis B Virus Nucleic Acid Detection Kit from Shanghai Fosun Diagnostics Co., Ltd. (Hereinafter Referred to as Fosun)

Fosun Hepatitis B Virus Quantitative Detection Kit was used. Product Name: Hepatitis B Virus Nucleic Acid Detection Kit (PCR-Fluorescence Probe Method), Medical Device Registration Certificate Number: 20173401101, approval for registration of medical devices in China. 23 samples of Example 6 were under detection, and the specific steps were carried out in accordance with the operating instructions of the kit. The detection results of samples 1-23 are shown in Table 2.

TABLE 2
Quantitative detection results of test samples

| Sample No. | Concentration (copies/mL) |
|---|---|
| Test Sample 1 | 2.95 × 10³ |
| Test Sample 2 | 2.21 × 10³ |
| Test Sample 3 | 1.54 × 10³ |
| Test Sample 4 | 3.60 × 10³ |
| Test Sample 5 | 2.23 × 10³ |
| Test Sample 6 | 3.95 × 10³ |
| Test Sample 7 | 9.87 × 10³ |
| Test Sample 8 | 3.52 × 10³ |
| Test Sample 9 | 6.30 × 10⁴ |
| Test Sample 10 | 3.97 × 10⁴ |
| Test Sample 11 | 1.13 × 10⁴ |
| Test Sample 12 | 7.70 × 10⁴ |
| Test Sample 13 | 1.25 × 10⁴ |
| Test Sample 14 | 1.47 × 10⁴ |
| Test Sample 15 | 2.19 × 10⁵ |
| Test Sample 16 | 1.21 × 10⁵ |
| Test Sample 17 | 1.27 × 10⁵ |
| Test Sample 18 | 5.85 × 10⁶ |
| Test Sample 19 | 3.55 × 10⁶ |
| Test Sample 20 | 2.49 × 10⁶ |
| Test Sample 21 | 5.69 × 10⁷ |
| Test Sample 22 | 1.80 × 10⁷ |
| Test Sample 23 | 2.64 × 10⁷ |

The independent HBV DNA positive samples were analyzed with the Hepatitis B Virus Nucleic Acid Detection Kit (Shanghai Fosun), and all the tested samples were HBV DNA positive. However, the method and the kit of the present disclosure have higher detection sensitivity, the concentration results from the quantitative detection of different gene fragments in most of the samples to be tested were higher than that of the Fosun kit. By simultaneously amplifying different gene fragments of the same sample, the occurrence of false negative results can be further reduced.

In summary, by designing the primers and probes on the conservative sequences of the hepatitis B virus S, C, and X gene regions, the present disclosure provides method of using triplex triple-gene fluorescence quantitative PCR technology to simultaneously detect HBV DNA in a single tube. The present disclosure has simple and rapid operation, improves the sensitivity and specificity of the hepatitis B virus detection method, minimizes the probability of false negative results due to mutations which further improves the detection efficiency, and is suitable for the detection of occult hepatitis B virus infection with low viral load.

The primers provided by the present disclosure have strong specificity and high sensitivity, and can be used for effective amplification of HBV DNA, thereby realizing accurate and quantitative detection of HBV infection. By simultaneous amplification and detection of highly conserved fragments of three different gene regions of HBV, the present disclosure effectively reduces the occurrence of problems such as false negatives, and improves the specificity of hepatitis B virus detection. The present disclosure has a fast detection speed requiring only one hour, and it requires a total of only 2˜3 hours including nucleic acid extraction. The present disclosure has simple steps, and enables the high-throughput sample detection at the same time. The present disclosure is suitable for clinical or laboratory quantitative detection of hepatitis B virus infection, monitoring and prediction of hepatitis B virus prevalence, and detecting and evaluation of curative effects timely.

The above-mentioned embodiments only exemplarily illustrate the principles and effects of the present disclosure, but are not used to limit the present disclosure. Anyone familiar with this technology can modify or change the above-mentioned embodiments without departing from the spirit and scope of the present disclosure. Therefore, all equivalent modifications or changes made by those with ordinary knowledge in the technical field without departing from the spirit and technical ideas disclosed by the present disclosure should still be encompasses by the claims of the present disclosure.

1. A primer and probe combination, selected from at least one of an S gene region primer and probe combination, a C gene region primer and probe combination, and an X gene region primer and probe combination;
wherein the S gene region primer and probe combination is specifically selected from any one of the following combinations:

| | Combination S1: |
|---|---|
| | an upstream primer comprising a sequence |
| | as shown in |
| SEQ ID NO: 1 | |
| | (TTGCCCGTTTGTCCTCTAATTC), |
| | a downstream primer comprising a sequence |
| | as shown in |
| SEQ ID NO: 2 | |
| | (CATCCATAGGTTTTGTACAGCAAC), |
| | and |
| | a probe comprising a sequence as shown in |
| SEQ ID NO: 3 | |
| | (AGGATCATCAACCACCAGCACGGG); |
| | Combination S2: |
| | an upstream primer comprising a sequence |
| | as shown in |
| SEQ ID NO: 4 | |
| | (GTGTCTGCGGCGTTTATCA), |
| | a downstream primer comprising a sequence |
| | as shown in |
| SEQ ID NO: 5 | |
| | (CCCGTTTGTCCTCTAATTCCAG), |
| | and |
| | a probe comprising a sequence as shown in |
| SEQ ID NO: 6 | |
| | (TTCCTCTGCATCCTGCTGCTATGCC); |
| | and |
| | Combination S3: |
| | an upstream primer comprising a sequence |
| | as shown in |
| SEQ ID NO: 7 | |
| | (TGCCCGTTTGTCCTCTAATTCC), |
| | a downstream primer comprising a sequence |
| | as shown in |
| SEQ ID NO: 8 | |
| | (AGGTGCAGTTTCCATCCATAGG), |
| | and |
| | a probe comprising a sequence as shown in |
| SEQ ID NO: 9 | |
| | (TCATCAACCACCAGCACGGGACCA); |

the C gene region primer and probe combination is specifically selected from any one of the following combinations:

| | Combination C1: |
|---|---|
| | an upstream primer comprising a sequence |
| | as shown in |
| SEQ ID NO: 10 | |
| | (AGCCTTAAAATCTCCTGAGCATTG), |
| | a downstream primer comprising a sequence |
| | as shown in |
| SEQ ID NO: 11 | |
| | (CAAATTATTACCCACCCAGGTAGC), |
| | and |
| | a probe comprising a sequence as shown in |
| SEQ ID NO: 12 | |
| | (TCACCACACAGCACTCAGGCAAGC); |
| | Combination C2: |
| | an upstream primer comprising a sequence |
| | as shown in |
| SEQ ID NO: 13 | |
| | (AGGCAGGTCCCCTAGAAGAAG), |
| | a downstream primer comprising a sequence |
| | as shown in |
| SEQ ID NO: 14 | |
| | (ACATTGGGATTCCCGAGATTGAG), |
| | and |
| | a probe comprising a sequence as shown in |
| SEQ ID NO: 15 | |
| | (ACTCCCTCGCCTCGCAGACGAAGG); |
| | and |
| | Combination C3: |
| | an upstream primer comprising a sequence |
| | as shown in |
| SEQ ID NO: 16 | |
| | (ATCAACACTTCCGGAAACTACTG), |
| | a downstream primer comprising a sequence |
| | as shown in |
| SEQ ID NO: 17 | |
| | (TTCCCGAGATTGAGATCTTCTGC), |
| | and |
| | a probe comprising a sequence as shown in |
| SEQ ID NO: 18 | |
| | (GGCAGGTCCCCTAGAAGAAGAACT); |

the X gene region primer and probe combination is specifically selected from any one of the following combinations:

| | Combination X1: |
|---|---|
| | an upstream primer comprising a sequence |
| | as shown in |
| SEQ ID NO: 19 | |
| | (TGCACTTCGCTTCACCTCTG), |
| | a downstream primer comprising a sequence |
| | as shown in |
| SEQ ID NO: 20 | |
| | (TTGCTGAAAGTCCAAGAGTCCTC), |
| | and |
| | a probe comprising a sequence as shown in |
| SEQ ID NO: 21 | |
| | (CGCATGGAGACCACCGTGAACGCC); |
| | Combination X2: |
| | an upstream primer comprising a sequence |
| | as shown in |
| SEQ ID NO: 22 | |
| | (ACTTCGCTTCACCTCTGCAC), |
| | a downstream primer comprising a sequence |
| | as shown in |
| SEQ ID NO: 23 | |
| | (AGGTCGGTCGTTGACATTGC), |
| | and |
| | a probe comprising a sequenceas shown in |
| SEQ ID NO: 24 | |
| | (AGACCACCGTGAACGCCCACCG); |
| | and |
| | Combination X3: |
| | an upstream primer comprising a sequence |
| | as shown in |
| SEQ ID NO: 25 | |
| | (CACCTCTCTTTACGCGGACTC), |
| | a downstream primer comprising a sequence |
| | as shown in |
| SEQ ID NO: 26 | |
| | (AGTCCTCTTATGCAAGACCTTGG), |
| | and |
| | a probe comprising a sequence as shown in |
| SEQ ID NO: 27 | |
| | (TGCCTTCTCATCTGCCGGACCGTG); |

each of the above probes comprises a fluorescent dye and a fluorescence quencher.

2. The primer and probe combination according to claim 1, wherein the fluorescent dye is selected from at least one of VIC, FAM, HEX, Cy5, Rox, and TET.

3. The primer and probe combination according to claim 1, wherein the fluorescence quencher is selected from at least one of BHQ-1, BHQ-2, BHQ-3, BBQ, and TAMRA.

4. (canceled)

5. (canceled)

6. A kit comprising the primer and probe combination according to claim 1.

7. The kit according to claim 6, further comprising a fluorescent quantitative reaction solution.

8. The kit according to claim 6, further comprising at least one of a template, a positive control substance, and a negative control substance.

9. The kit according to claim 8, wherein the template is selected from any one of a standard positive template and a human genomic DNA extract.

10. The kit according to claim 8, wherein the positive control substance is hepatitis B virus DNA, and the negative control substance is water.


SEQUENCE LISTING (30 sequences)

SEQ ID NO: 1 - DNA, 22 bp, Artificial Sequence
  S1 forward primer
ttgcccgtttgtcctctaattc

SEQ ID NO: 2 - DNA, 24 bp, Artificial Sequence
  S1 reverse primer
catccataggttttgtacagcaac

SEQ ID NO: 3 - DNA, 24 bp, Artificial Sequence
  S1 probe
aggatcatcaaccaccagcacggg

SEQ ID NO: 4 - DNA, 19 bp, Artificial Sequence
  S2 forward primer
gtgtctgcggcgtttatca

SEQ ID NO: 5 - DNA, 22 bp, Artificial Sequence
  S2 reverse primer
cccgtttgtcctctaattccag

SEQ ID NO: 6 - DNA, 25 bp, Artificial Sequence
  S2 probe
ttcctctgcatcctgctgctatgcc

SEQ ID NO: 7 - DNA, 22 bp, Artificial Sequence
  S3 forward primer
tgcccgtttgtcctctaattcc

SEQ ID NO: 8 - DNA, 22 bp, Artificial Sequence
  S3 reverse primer
aggtgcagtttccatccatagg

SEQ ID NO: 9 - DNA, 24 bp, Artificial Sequence
  S3 probe
tcatcaaccaccagcacgggacca

SEQ ID NO: 10 - DNA, 24 bp, Artificial Sequence
  C1 forward primer
agccttaaaatctcctgagcattg

SEQ ID NO: 11 - DNA, 24 bp, Artificial Sequence
  C1 reverse primer
caaattattacccacccaggtagc

SEQ ID NO: 12 - DNA, 24 bp, Artificial Sequence
  C1 probe
tcaccacacagcactcaggcaagc

SEQ ID NO: 13 - DNA, 21 bp, Artificial Sequence
  C2 forward primer
aggcaggtcccctagaagaag

SEQ ID NO: 14 - DNA, 23 bp, Artificial Sequence
  C2 reverse primer
acattgggattcccgagattgag

SEQ ID NO: 15 - DNA, 24 bp, Artificial Sequence
  C2 probe
actccctcgcctcgcagacgaagg

SEQ ID NO: 16 - DNA, 23 bp, Artificial Sequence
  C3 forward primer
atcaacacttccggaaactactg

SEQ ID NO: 17 - DNA, 23 bp, Artificial Sequence
  C3 reverse primer
ttcccgagattgagatcttctgc

SEQ ID NO: 18 - DNA, 24 bp, Artificial Sequence
  C3 probe
ggcaggtcccctagaagaagaact

SEQ ID NO: 19 - DNA, 20 bp, Artificial Sequence
  X1 forward primer
tgcacttcgcttcacctctg

SEQ ID NO: 20 - DNA, 23 bp, Artificial Sequence
  X1 reverse primer
ttgctgaaagtccaagagtcctc

SEQ ID NO: 21 - DNA, 24 bp, Artificial Sequence
  X1 probe
cgcatggagaccaccgtgaacgcc

SEQ ID NO: 22 - DNA, 20 bp, Artificial Sequence
  X2 forward primer
acttcgcttcacctctgcac

SEQ ID NO: 23 - DNA, 20 bp, Artificial Sequence
  X2 reverse primer
aggtcggtcgttgacattgc

SEQ ID NO: 24 - DNA, 22 bp, Artificial Sequence
  X2 probe
agaccaccgtgaacgcccaccg

SEQ ID NO: 25 - DNA, 21 bp, Artificial Sequence
  X3 forward primer
cacctctctttacgcggactc

SEQ ID NO: 26 - DNA, 23 bp, Artificial Sequence
  X3 reverse primer
agtcctcttatgcaagaccttgg

SEQ ID NO: 27 - DNA, 24 bp, Artificial Sequence
  X3 probe
tgccttctcatctgccggaccgtg

SEQ ID NO: 28 - DNA, 681 bp, Artificial Sequence
  S gene region
atggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaa

SEQ ID NO: 29 - DNA, 552 bp, Artificial Sequence
  C gene region
atggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttag

SEQ ID NO: 30 - DNA, 465 bp, Artificial Sequence
  X gene region
atggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaa